(19) United States
(12) Patent Application Publication
(10) Pub. No.: US 2003/0134774 A1
(43) Pub. Date: Jul. 17, 2003

(54) Methods for inhibiting cardiac disorders

(76) Inventors: Susan F. Steinberg, Bronx, NY (US); Abdelkarim Sabri, Birmingham, AL (US)

Correspondence Address: Cooper and Dunham LLP, 1185 Avenue of the Americas, New York, NY, 10036, US

(21) Appl. No.: 10/172,696

(22) Filed: Jun. 14, 2002

Related U.S. Application Data
(60) Provisional application No. 60/298,509, filed on Jun. 15, 2001.

(51) Int. Cl. A61K 31/00

(52) U.S. Cl. 514/1

(57) ABSTRACT

This invention provides methods of (1) inhibiting the onset of a cardiac disorder in a subject afflicted with cardiac hypertrophy, (2) reducing the activity of PKC-δ or PKC-ε present in cardiomyocytes of a subject afflicted with cardiac hypertrophy, and (3) reducing the activity of PKC-δ or PKC-ε in a hypertrophic cardiomyocyte by administering an agent that specifically reduces the activity of PKC-δ or PKC-ε present therein. This invention also provides an article of manufacture inhibiting the onset of a cardiac disorder in a subject afflicted with cardiac hypertrophy.

The invention described herein was made with government support under U.S.P.H.S.-N.H.L.B.I. grant HL-64639 (S.F.S.) and NIH/NIAID grant AI38396 (B.A.W.). Accordingly, the United States government has certain rights in this invention.

Throughout this application, various references are cited. Disclosure of these references in their entirety is hereby incorporated by reference into this application to more fully describe the state of the art to which this invention pertains.

### BACKGROUND OF THE INVENTION

Cardiac Hypertrophy

Myocardial hypertrophy, also called cardiac hypertrophy, is an adaptive response to stresses that increase cardiac work (28-30). The resulting increase in tissue mass diminishes systolic wall stress and improves contractile performance in the short term. However, compensated hypertrophy generally progresses to decompensated cardiac failure with chamber dilatation and contractile dysfunction.

Since cardiac failure represents a major public health problem with substantial mortality, many laboratories have invested considerable effort to understand the regulatory determinants that contribute to the development of hypertrophy and its transition to heart failure.

The Role of Gαq in Cardiac Hypertrophy

The focus of many recent studies has been on the family of transmembrane-spanning receptors that activate Gq proteins. These receptors include, for example, the receptors for endothelin, α1-adrenergic agonists, angiotensin II, prostaglandin F_{2α} and thrombin. Agonist stimulation or transgenic cardiac-specific overexpression of Gq-coupled G protein-coupled receptors (GPCRs) leads to cardiac hypertrophy with many common molecular and morphologic features. This form of hypertrophy generally is attributed to Gq protein α subunit activation of effector pathways that individually have been implicated in hypertrophic cardiomyocyte growth responses (i.e., protein kinase C [PKC], mitogen-activated protein kinase [MAPK] cascades, tyrosine kinases).

However, the precise cellular actions of Gαq proteins are not readily resolved by studies of Gq-coupled GPCRs for two major reasons. First, many GPCRs that couple to Gq also activate other G protein classes, perhaps explaining subtle differences in resultant hypertrophic phenotypes (1,2). Second, heterotrimeric Gq protein activation by GPCRs liberates βγ dimers, which mobilize distinct downstream signaling targets that may be critical for full expression of the molecular and morphological features of cardiomyocyte hypertrophy. Indeed, the prediction that Gαq subunits activate only a subset of the signaling machinery recruited by agonist-occupied GPCRs is borne out by experimental evidence that Gαq subunits alone are not sufficient to induce the entire spectrum of morphologic changes characteristically induced by hypertrophic agonists acting at their cognate GPCRs (3).

Previous studies have used strategies more directly targeted to Gαq to resolve its role in cardiomyocyte hypertrophy.

Initial studies demonstrated that microinjection of inhibitory antibodies to Gα_{q/11} in rat cardiomyocyte cultures or cardiac-restricted overexpression of a Gαq inhibitory peptide (which prevents signal transmission at the receptor-Gαq subunit interface) in mice interferes with the acquisition of features of (a) cardiac hypertrophy in response to α1-adrenergic receptors or (b) pressure overload hypertrophy (4,5). Subsequently, the consequences of Gα subunit activation in cardiomyocytes were delineated by overexpressing Gαq subunits in cardiomyocyte cultures or genetically targeting the Gαq transgene to mouse myocardium.

These studies provided the unanticipated evidence that modest increases in wild-type Gαq expression induce stable cardiac hypertrophy, but very intense Gαq stimulation (with very high levels of wild-type Gαq proteins or the constitutively activated mutant form of the Gαq) induces a dilated cardiomyopathy, with evidence of functional decompensation and cardiomyocyte apoptosis (6-8).

These observations suggest that hypertrophy and apoptosis may represent different phases of the same process, initiated by a common Gαq-activated biochemical signal. However, a precise mechanism has not been described whereby the traditional targets of Gαq subunits that induce hypertrophy can also act as triggers for the development of cardiomyocyte apoptosis. Moreover, the relative importance of apoptosis as a consequence of Gαq signaling in the physiologic context remains uncertain, since apoptosis is detected only in the context of molecular strategies with intense Gαq stimulation (with potentially altered stoichiometry of Gαq subunits to downstream signaling partners and/or aberrant Gαq targeting to the plasma membrane).

The Role of PKC-δ and PKC-ε in Cardiac Disorders

Prior studies have used a peptide-based strategy to either inhibit or activate the epsilon and delta isoforms of PKC (i.e., PKC-δ and PKC-ε), and thereby investigate the role of these PKC isoforms in certain cardiac disorders. The bulk of the studies to date have examined the role of epsilon and delta PKC in ischemic preconditioning, a process whereby brief exposures to ischemia protect the heart from subsequent prolonged ischemic insults. This phenomenon is widely accepted, as is the role of PKC in this process (31, 32). However, the tools needed to define the role of individual PKC isoforms had not previously been available.

Recent studies report the use of PKC epsilon/delta peptide inhibitors and activators to address this issue, describing distinct effects of epsilon and delta PKC on early and late phases of the preconditioning processes. Published studies have focused on the cardioprotection that comes from ingesting small amounts of ethanol. By perfusing PKC activator and inhibitor peptides into adult mouse hearts, ethanol-dependent protection is mediated (at least in part) by PKCε, whereas PKCδ induces further cardiac damage (33, 34).

Other studies have used a molecular approach to administer PKCε inhibitor or activator peptides. There, expression of the peptide inhibitor or activator is driven by the myosin heavy chain promoter. Hence, the peptides are synthesized only in cardiomyocytes, starting at the time of birth. This method permits studying the effect of PKCε activation or inhibition on normal postnatal growth and development of the heart. In this manner, PKCε activation was implicated in the normal physiological growth of the heart during development (35).

Peptide inhibitors have never been used to examine the role of PKCε or PKCδ in modulating the progression of heart failure and/or its transition to failure.

Additional studies have used antisense oligonucleotides to inhibit PKC isoform activity (27, 36-38). However, antisense oligonucleotides have not been used to examine the role of PKCε or PKCδ in modulating the progression of heart failure and/or its transition to failure.

In sum, the biochemical pathway which mediates the transition from cardiomyocyte hypertrophy to apoptosis is not understood. Elucidating this pathway would be useful in identifying drug targets for preventing cardiac disorders.

### SUMMARY OF THE INVENTION

This invention provides a method of inhibiting the onset of a cardiac disorder in a subject afflicted with cardiac hypertrophy, comprising administering to the subject a prophylactically effective amount of an agent that specifically reduces the activity of PKC-δ or PKC-ε present in the subject's cardiomyocytes.

This invention also provides a method of reducing the activity of PKC-δ or PKC-ε present in cardiomyocytes of a subject afflicted with cardiac hypertrophy, comprising administering to the subject an effective amount of an agent that specifically reduces the activity of PKC-δ or PKC-ε present in the subject's cardiomyocytes.

This invention further provides a method of reducing the activity of PKC-δ or PKC-ε in a hypertrophic cardiomyocyte, comprising contacting the cardiomyocyte under suitable conditions with an agent that specifically reduces the activity of PKC-δ or PKC-ε present therein.

Finally, this invention provides an article of manufacture comprising (a) an agent that specifically reduces the activity of PKC-δ or PKC-ε present in a cardiomyocyte, and (b) instructions for using the agent to inhibit the onset of a cardiac disorder in a subject afflicted with cardiac hypertrophy.

### BRIEF DESCRIPTION OF THE FIGURES

FIG. 1

rPMT activates PLC. Cardiomyocyte cultures were incubated in media containing 3 μCi/ml [³H] myoinositol for 96 hr, with 400 ng/ml rPMT for the indicated intervals at the end of this interval (Panel A) or the indicated concentration of rPMT included in the culture medium during the final 24 hr of culture (Panel B). This was followed by 30 min in the presence of 10 mM LiCl to inhibit the hydrolysis of inositol phosphate metabolites. Results are expressed as cpm over basal (mean±SEM; n=3) from a single experiment, and are representative of results obtained in 3 separate culture preparations.

FIG. 2

rPMT selectively activates nPKC isoforms. Cardiomyocyte cultures were incubated in culture medium without or with NE (10 μM), rPMT (400 ng/ml) or PMA (100 nM) for 24 hr (Panels A and C) or 5 min (Panel B). Soluble extracts were prepared and separated by SDS-PAGE followed by immunodetection of total PKC isoform abundance with antibodies that are specific for the individual PKC isoforms (Panel A), or phosphorylated forms of PKC (largely PKCε) with a pan-phospho-PKC antibody (Panels B and C), as described in Methods below. GF109203 was included at 5 μM 30 min prior to stimulation as indicated.

FIG. 3

rPMT promotes the activation of ERK1/2, JNK and p38-MAPK cascades. Incubations were for 24 hr in the presence of the indicated concentrations of rPMT. PMA (100 nM, 5 min) and sorbitol (0.5 mM, 5 min) were included as positive controls. Cell lysates were subjected to SDS-PAGE followed by Western blotting as described in Methods below. Phosphorylated forms of ERK1/2 (A), p38 MAP kinase (B) or JNK (C) were detected by specific anti-phospho-ERK1/2, -p38 MAP kinase or -JNK antibodies, respectively. Blots were stripped and subjected to immunoblot analysis with total ERK1/2 or p38 MAP kinase to normalize for minor variations in protein loading (Panels A and B). Top: representative autoradiograms (with each lane from a single gel exposed for the same duration). Bottom: quantification of each series of experiments (n=3).

FIG. 4

rPMT mimics the effect of norepinephrine to promote cardiomyocyte hypertrophy. Cardiomyocytes were cultured in serum-free medium without or with rPMT (400 ng/ml) or norepinephrine (NE, 10 μM) for 48 hr. Cardiomyocytes were fixed, permeabilized, and stained with monoclonal anti-α-actinin sarcomeric (Panel A, magnification×100) or subjected to Northern blot analysis for measurements of steady-state ANF mRNA expression (Panel B). Effects of rPMT (black bars) and NE (gray bars) vs. vehicle (white bars) on sarcomeric organization (Panel C), cell size (Panel D), and protein synthesis (Panel E) are illustrated (n=3 for each).

FIG. 5

rPMT transactivation of the EGF receptor in cardiac fibroblasts, but not cardiomyocytes. Neonatal rat cardiomyocytes (Panel A) and cardiac fibroblasts (Panel B) were treated with rPMT (400 ng/ml) for 24 hr or EGF (50 ng/ml) for 5 min without or with AG1478 (2 μM, starting 30 min prior to stimulation). Cell lysates were subjected to SDS-PAGE followed by Western blotting with the anti-phospho-ERK1/2 antibody. Data are from a representative gel, with similar results obtained in 3 separate experiments.

FIG. 6

rPMT decreases basal AKT phosphorylation and prevents its phosphorylation by EGF. Neonatal rat cardiomyocytes were treated with rPMT (400 ng/ml) for 24 hr (Panel A), 48 hr (Panel B) or with EGF (50 ng/ml) for 5 min. Cell lysates were subjected to SDS-PAGE followed by Western blotting with antibodies that recognize the phosphorylated form or total AKT. Data are from representative gels, with similar results obtained in 3 separate experiments.

FIG. 7

rPMT activation of PKC negatively regulates AKT.

Panel A: Cells were treated with norepinephrine (NE, 10 μM) or PMA (100 nM) for 5 min without or with GF109203 (5 μM).

Panel B: Cells were treated with rPMT (400 ng/ml), NE (10 μM) or PMA (100 nM) for 24 hr without or with Go6983 (5 μM). Go6983 (rather than GF109203) was used in experiments that were carried out for 24 hr, since it completely inhibited PMA-dependent down-regulation of PKC isoforms, whereas GF109203 did not (see text). Cell lysates were subjected to SDS-PAGE followed by Western blotting using a specific anti-phospho-AKT antibody. Data are from representative gels, with similar results obtained in 3 separate culture preparations.

Panel C: Quantification of the rPMT-dependent decrease of AKT phosphorylation. Data are mean±SEM from 3 independent experiments. *<0.05 vs control.

FIG. 8

rPMT increases H₂O₂-induced apoptosis in cardiomyocyte cultures. Cells were treated without or with rPMT (400 ng/ml) for 24 hr followed by H₂0₂ (500 μM) for 24 hr. The percentage of cells undergoing apoptosis was measured by TUNEL assay as described in Methods below. Data are from a single experiment and are representative of results obtained on 3 separate culture preparations.

FIG. 9

Schematic of rPMT signaling pathways in rat neonatal cardiomyocytes. rPMT induces sustained Gαq activation which stimulates nPKC isoforms; nPKC negatively regulates the AKT survival pathway.

### DETAILED DESCRIPTION OF THE INVENTION

Definitions

As used in this application, except as otherwise expressly provided herein, each of the following terms shall have the meaning set forth below.

As used herein, “cardiac disorder” includes, without limitation, any disorder characterized by the inhibition by PKC-δ and/or PKC-ε of the AKT-activated cell survival pathway in the cardiomyocytes of an afflicted subject. Examples of cardiac disorders include hypertensive heart disease, valvular heart disease (e.g., aortic stenosis), diabetic heart disease and ischemic heart disease.

As used herein, “cardiac hypertrophy” shall mean the enlargement of cardiac tissue due to the enlargement of cardiomyocytes therein. Causes of cardiomyocyte enlargement include, without limitation, mechanical stress, physiological stress, sarcomeric increase, and re-induction of fetal gene expression. “Cardiomyocyte” and “cardiac cell” are used equivalently herein.

“DNAzyme” shall mean a catalytic nucleic acid molecule which is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain. In one embodiment, the DNAzyme cleaves RNA molecules, and is of the “10-23” model, having the catalytic domain GGCTAGCTACAACGA. This type of DNAzyme is described in the art.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Nucleic acid molecule” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA). Nucleic acid molecules include, without limitation, anti-sense molecules and catalytic nucleic acid molecules such as ribozymes and DNAzymes.

As used herein, “reducing” the activity of PKC-δ or PKC-ε in a cardiomyocyte includes, without limitation, (a) reducing the quantity of PKC-δ or PKC-ε present therein, via inhibiting transcription and/or translation thereof; (b) reducing the quantity of PKC-δ or PKC-ε properly translocated therein; and (c) reducing the quantity of active PKC-δ or PKC-ε therein, via inhibiting PKC-δ or PKC-ε activation and/or interfering with PKC-δ or PKC-ε catalytic activity. PKC-δ or PKC-ε “activity” shall mean the phosphorylation by PKC-δ or PKC-ε of a target substrate thereof. In one embodiment of the invention, the reduction of activity is with respect to the ability of PKC-δ or PKC-ε to phosphorylate all target substrates thereof. In another embodiment, the reduction of activity is with respect to the ability of PKC-δ or PKC-ε to phosphorylate one target substrate thereof.

“Ribozyme” shall mean a catalytic nucleic acid molecule which is RNA or whose catalytic component is RNA, and which specifically recognizes and cleaves a distinct target nucleic acid sequence (also referred to herein as a “target” or “target sequence”), which can be either DNA or RNA. Each ribozyme has a catalytic component (also referred to as a “catalytic domain”) and a target sequence-binding component consisting of two binding domains, one on either side of the catalytic domain. Ribozymes generally are described in the literature. In the preferred embodiment, the ribozyme is a hammerhead ribozyme.

As used herein, an agent that “specifically reduces PKC-δ activity” shall mean an agent that reduces the activity of PKC-δ more than it reduces the activity of any other PKC isoform. Likewise, an agent that “specifically reduces PKC-ε activity” shall mean an agent that reduces the activity of PKC-ε more than it reduces the activity of any other PKC isoform. An agent that “specifically reduces PKC-δ and PKC-ε activity” shall mean an agent that reduces the activity of both PKC-δ and PKC-ε more than it reduces the activity of any other PKC isoform.

As used herein, “subject” shall include, without limitation, a mammal such as a mouse, a rat, a dog, a guinea pig, a ferret, a rabbit or a primate.

As used herein, “suitable conditions” for activating a cardiomyocyte with an agent that specifically reduces the activity of PKC-δ or PKC-ε present therein shall mean any condition which permits the agent to enter the cardiomyocyte and to specifically inhibit the PKC-δ or PKC-ε. Suitable conditions include, for example, physiological conditions.

Embodiments of the Invention

This invention is based on applicants' surprising discovery of the biochemical pathway that mediates the transition from cardiomyocyte hypertrophy to apoptosis. Through this discovery, applicants have shown, among other things, that PKCδ and PKCε play a key role in this transition, and hence, that their inhibition permits the prophylaxis of cardiac disorders in hypertrophic subjects.

Accordingly, this invention provides a method of inhibiting the onset of a cardiac disorder in a subject afflicted with cardiac hypertrophy, comprising administering to the subject a prophylactically effective amount of an agent that specifically reduces the activity of PKC-δ or PKC-ε present in the subject's cardiomyocytes.

This invention also provides a method of reducing the activity of PKC-δ or PKC-ε present in cardiomyocytes of a subject afflicted with cardiac hypertrophy, comprising administering to the subject an effective amount of an agent that specifically reduces the activity of PKC-δ or PKC-ε present in the subject's cardiomyocytes.

In one embodiment of these methods, the subject is selected from the group consisting of a mouse, a rat, a dog, a guinea pig, a ferret, a rabbit and a primate. In a preferred embodiment, the subject is a human. Further, the agent can specifically inhibit PKC-δ, PKC-ε, or both. The agent can be a nucleic acid, a polypeptide or rottlerin.

Determining a prophylactically effective amount of a pharmaceutical composition can be done based on animal data using routine computational methods. In one embodiment, the prophylactically effective amount contains between about 0.1 mg and about 1 g of agent (e.g., nucleic acid or peptide) in the pharmaceutical composition. In another embodiment, the effective amount contains between about 1 mg and about 100 mg of agent. In a further embodiment, the effective amount contains between about 10 mg and about 50 mg of agent, and preferably about 25 mg thereof.

In this invention, administering the instant pharmaceutical composition can be effected or performed using any of the various methods and delivery systems known to those skilled in the art. The administering can be performed, for example, intravenously, pericardially, orally, via implant, transmucosally, transdermally, intramuscularly, and subcutaneously. Such administration can be performed, for example, once, a plurality of times, and/or over one or more extended periods. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules.

These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmityl-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP (N[1-(2,3-dioleoyloxy)-N,N,N-trimethyl ammoniummethylsulfate) (Boehringer Manheim); and (4) Lipofectamine, 3:1 (M/M) liposome formulation of the polycationic lipid DOSPA and the neutral lipid DOPE (GIBCO BRL).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

This invention further provides a method of reducing the activity of PKC-δ or PKC-ε in a hypertrophic cardiomyocyte, comprising contacting the cardiomyocyte under suitable conditions with an agent that specifically reduces the activity of PKC-δ or PKC-ε present therein.

In one embodiment, the agent specifically inhibits PKC-δ. In another embodiment, the agent specifically inhibits PKC-ε. In a further embodiment, the agent inhibits both PKC-δ and PKC-ε. The agent can be a nucleic acid, a polypeptide or rottlerin.

Finally, this invention provides an article of manufacture comprising (a) an agent that specifically reduces the activity of PKC-δ or PKC-ε present in a cardiomyocyte, and (b) instructions for using the agent to inhibit the onset of a cardiac disorder in a subject afflicted with cardiac hypertrophy.

The embodiments with respect to the agent and its specificity are as per the instant methods. The article of manufacture may further comprise a pharmaceutically acceptable carrier. Examples of the instant article of manufacture include a pre-filled, labeled vial.

This invention will be better understood from the Experimental Details that follow. However, one skilled in the art will readily appreciate that the specific methods and results discussed are merely illustrative of the invention as described more fully in the claims which follow thereafter.

Experimental Details

Observations by others suggest that hypertrophy and apoptosis may represent different phases of the same process, initiated by a common Gαq-activated biochemical signal. The precise mechanism(s) whereby traditional targets of Gαq subunits that induce hypertrophy also trigger cardiomyocyte apoptosis is not clear, and is explored here with recombinant Pasteurella multocida toxin (rPMT, a Gαq agonist).

Pasteuralla multocida is a veterinary respiratory pathogen and an extremely potent fibroblast cell mitogen. The pathogenicity of Pasteuralla multocida derives from its protein toxin, which is sufficient to reproduce all of the major disease symptoms in experimental animals. Pasteuralla multocida toxin (PMT) is internalized via receptor-mediated endocytosis and acts intracellularly to activate signal transduction pathways including the hydrolysis of membrane inositol phospholipids to form IP₃ and diacylglycerol, mobilization of intracellular calcium, translocation of PKC, activation of the extracellular-regulated kinase [ERK] MAPK cascade, tyrosine phosphorylation of focal adhesion kinase (FAK) and paxillin, and enhanced actin stress fiber formation and focal adhesion assembly (9, 10).

On the basis of studies that show inhibition of recombinant PMT's (rPMT's) actions in Xenopus oocytes by antibodies directed against the α subunit of G_{q/11} or Gαq antisense RNA (11) or in HEK293 cells by overexpression of the C-terminal peptide inhibitor of G_{q/11} (9), the target of rPMT's actions has been identified as the free monomeric Gα_{q/11} subunit [with recent studies in fibroblasts deficient in either Gα_{q} or Gα₁₁ subunits localizing the rPMT-dependent activation of phospholipase C (PLC) to Gα_{q}, and not Gα₁₁ (12)]. Accordingly, this study uses rPMT as a pharmacological probe to elucidate the biochemical and functional consequences of endogenous Gα_{q} protein activation in cardiomyocytes.

Synopsis of Findings

Cells chronically cultured with rPMT display cardiomyocyte enlargement, sarcomeric organization, and increased ANF expression in association with activation of phospholipase C, novel protein kinase C (nPKC) isoforms (i.e., PKCδ and PKCε), ERK, and, to a lesser extent, JNK/p38-MAPK. rPMT stimulates the ERK cascade via epidermal growth factor (EGF) receptor transactivation in cardiac fibroblasts, but not in cardiomyocytes. Surprisingly, rPMT, or PKC activation by PMA, decreases basal AKT phosphorylation and prevents AKT phosphorylation by EGF. The rPMT-dependent decrease in AKT phosphorylation is abrogated by PKC inhibitors and is functionally significant; cardiomyocyte apoptosis is augmented in rPMT-treated cultures.

These results link nPKC isoform activation by Gαq to reduced AKT phosphorylation, impaired AKT stimulation by survival pathways, and enhanced susceptibility to apoptosis. AKT inhibition by PKC contributes to the transition from hypertrophy to heart failure. Hence, these results reveal PKCδ and PKCε as excellent targets for inhibiting the onset of apoptosis in hypertrophic cardiomyocytes.

I. Methods

A. Reagents

Antibodies and reagents were from the following sources: phospho-ERK1/2, total and phospho-p38 MAP kinase, phospho-JNK, total and phospho-AKT, and phospho-pan-PKC (Cell Signaling Technology); ERK1/2 (Santa Cruz Biotechnology); PKC-α and PKC-δ (Gibco-BRL); PKC-ε (generously provided by Dr. Doriano Fabbro, CIBA-GEIGY, Basel Switzerland, although this agent is publically available); and AG1478, Go6983 and GF109203 (Cabiochem). All other chemicals were obtained from standard commercial sources.

B. Preparation of Cultured Neonatal Rat Ventricular Cardiomyocytes

Cardiac myocytes were dissociated from the ventricles of two-day-old Wistar rats by a trypsin digestion protocol which incorporates a differential attachment procedure to enrich for cardiac myocytes, as described previously (13). For some experiments, attached cells were maintained as fibroblast cultures. For studies of inositol phosphates, MAPKs, AKT, and PKC cardiomyocytes were plated at a density of 5×10⁵ cells per ml (2 ml per 35-mm dish; 1 ml per 22.1-mm dish) and were cultured in MEM supplemented with 10% fetal calf serum. For the analysis of cell growth responses, cells were plated at a lower density (2.5×10⁵ cells per ml), to permit morphometric analysis of individual cells. Following incubation in 10% fetal calf serum overnight, the cells were washed and incubated in 1:1 DMEM/F-12 medium with no additions or test agents as indicated. Although the culturing technique includes a preplating step, which effectively decreases fibroblast contamination, cardiomyocytes grown in 10% fetal calf serum (but not serum-free medium, which itself curtails fibroblast growth) were subjected to 30 Gy of X-rays 24 hours after culture to halt the proliferative potential of any residual fibroblasts.

C. Inositol Phosphate Production

Cardiomyocytes grown in six-well plates were labeled with 3 μCi/ml [³H]myoinositol for 96 hr. Treatment with rPMT was during the final 24 hr of this interval. Cells were washed to remove unincorporated radioisotope and then incubated for an additional 20 min with HEPES-buffered saline containing 10 mM LiCl. Cells were lysed with 1.05 mls of chloroform/methanol/6M HCl (500:1000:3) and harvested, and lipids were extracted for 30 min at room temperature. 1.05 mls of chloroform/water (1:2) were added, and the mixture was vortexed and centrifuged at 2000 g for 5 min to separate the phases. The aqueous phase was transferred to Dowex anion-exchange columns, and inositol phosphates were eluted sequentially according to standard methods as described previously (13).

D. Immunoblotting for PKC Isoforms, ERK, p38-MAPK, JNK and AKT

Cells were maintained in culture in the presence of 10% FCS for 96 hr and then switched to serum-free medium for 24 hr prior to the addition of agonists as indicated. For PKC, extracts were prepared and subjected to SDS-PAGE to resolve individual isoforms according to methods described previously (14). It should be noted that norepinephrine increases protein recovery from cardiomyocytes cultured in serum-free medium for 72 hr, but protein recovery is not altered when norepinephrine (NE) treatment is in FCS [(13) and data not shown]. Hence, changes in PKC isoform abundance cannot be attributed to differences in total protein recovery between samples.

Specific immunoreactivity for individual PKC isoforms was quantified according to methods described previously. For assays of MAPKs and AKT, cells were exposed to test agents as indicated in individual experimental protocols, washed three times with ice-cold calcium/magnesium-free Dulbecco's PBS (pH 7.1), scraped into ice-cold extraction buffer [20 mM β-glycerophosphate pH 7.5, 20 mM sodium fluoride, 2 mM EDTA, 0.2 mM sodium vanadate, 10 μg/ml aprotinin, 25 μg/ml leupeptin, 50 μg/ml PMSF, and 0.3% (v/v) β-mercaptoethanol], lysed by sonication, and centrifuged at 10,000 g for 10 min at 4° C. The supernatant was diluted in SDS-PAGE sample buffer, boiled for 5 min, and stored at −70° C. Western blot analysis was performed with antibodies that are selective for the phosphorylated (activated) forms of ERK1/2, p38-MAPK, JNK, AKT, and pan-PKC according to manufacturer's instructions. For each panel in each Figure, the results are from a single gel exposed for a uniform duration. Bands were detected by enhanced chemiluminescence and blots were quantified by laser scanning densitometry.

E. Measurements of Cardiomyocyte Growth

Cardiomyocyte growth was assessed by measuring cell surface area by digitized image analysis. For each experimental point, 7-10 frames/dish were recorded at 40× magnification with a video camera attached to a Nikon microscope which was calibrated with a micrometer.

As a second index of cardiomyocyte growth, relative rates of protein synthesis were measured as radiolabeled phenylalanine incorporation into cell protein. Cells were stimulated in serum-free medium with agonists (or vehicle as control) for 48 hr at 37° C. The medium was replaced with serum-free medium containing [³H]phenylalanine (0.1 μCi/ml) and non-radioactive phenylalanine (0.3 mM, to minimize variations in the specific activity of the precursor pool responsible for protein synthesis) during the final 24 hr of stimulation. Cells were rinsed with PBS and incubated in 10% trichloroacetic acid for 30 min on ice. Cell precipitates were then washed twice with ice-cold 10% trichloroacetic acid and solubilized in 1% SDS (1 ml/well) at 37° C. for 1 hr. Aliquots of the SDS-soluble protein were counted in 5 ml of scintillant.

F. Northern Blot Analysis

Total RNA from neonatal rat cardiomyocytes were isolated by using a Qiagen kit according to manufacturer's instructions. A rat ANF cDNA probe (˜600 bp) was labeled with [³²P]dCTP. 10 μ/g of total RNA was separated on a 1% agarose gel and transferred to a nylon membrane (Amersham). Prehybridization and hybridization were performed according to standard methods. Normalization of signals was performed with a glyceraldehyde-3-phosphate dehydrogenase probe. Signals were detected by Phosphorimage analysis.

G. Immunocytochemistry

Cardiomyocytes were grown on slides precoated with fibronectin. After treatment, cells were fixed with cold methanol and permeabilized with 0.1% Triton X-100 and 0.2% BSA. Cells were incubated with α-actinin monoclonal antibody overnight at 4° C. followed by anti-mouse IgG-Cy3-labeled antibody for 30 min at room temperature. Fluorescence microscopy images were obtained at 100× magnification.

H. Terminal deoxynucleotidyl transferase-mediated dUTP nick-end labeling (TUNEL)

TUNEL staining was performed for detection of apoptotic cells according to manufacturer's instructions (Boehringer). Approximately 500 cardiomyocytes were imaged by fluorescence microscopy and the number of cells that scored TUNEL-positive is presented as a percentage of total.

II. Results

A. rPMT stimulates PLC, nPKC isoforms, and MAPK cascades

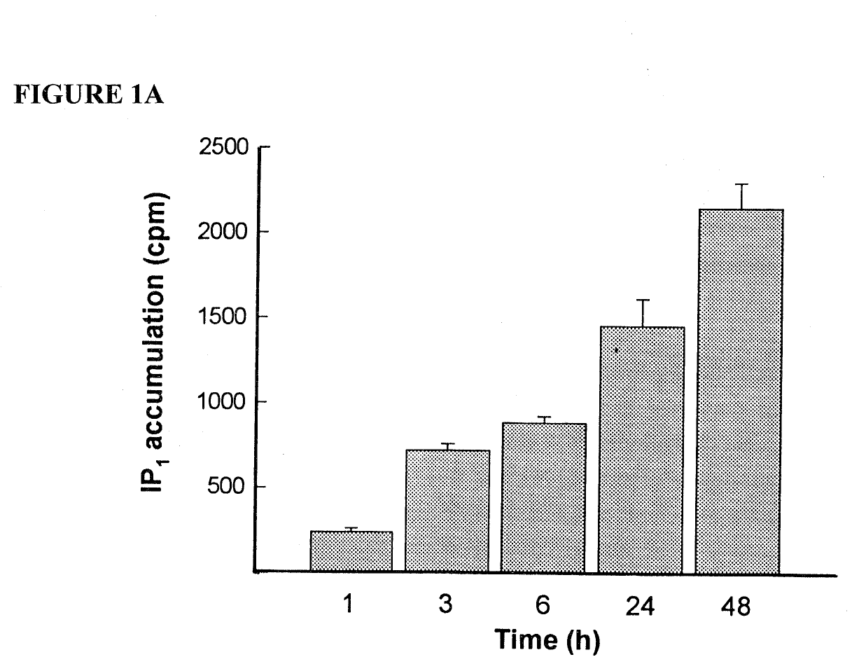

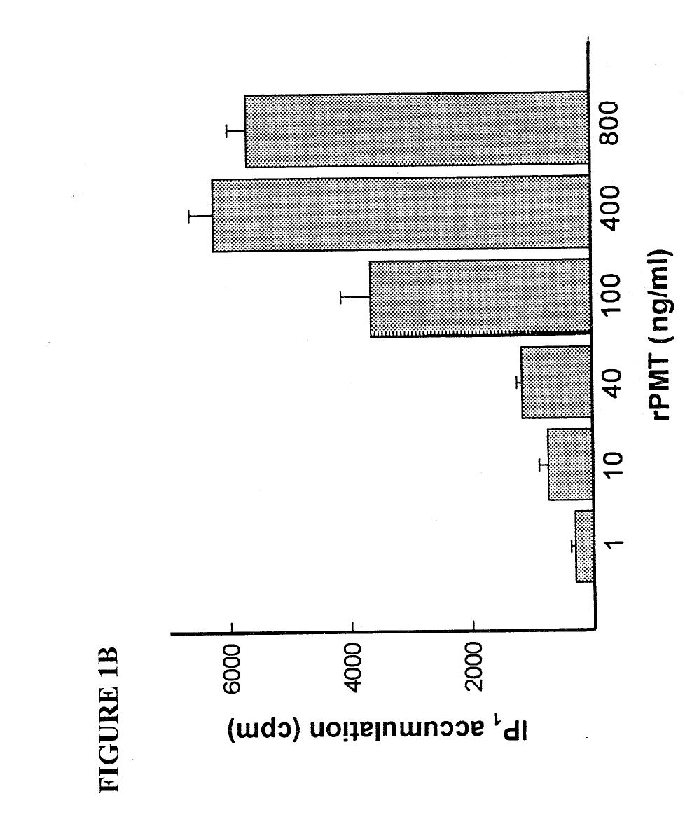

Consistent with previous evidence that rPMT gains access to the cell slowly, rPMT promotes inositol phosphate accumulation in cardiomyocytes, but with slow kinetics. The response is detectable at 1 hr and maximal at 48 hr (FIG. 1A). FIG. 1B shows the dose-dependence for inositol phosphate accumulation in response to rPMT. Maximal activation at 24 hr typically is with 400 ng/ml toxin, with individual batches of toxin displaying some variability.

rPMT-dependent activation of PLC also leads to the formation of diacylglycerol, the endogenous activator of PKC. There is general consensus that rat neonatal ventricular cardiomyocytes co-express multiple PKC isoforms, including calcium-sensitive PKCα, novel PKCδ and PKCε, and atypical PKCλ. A PKCζ is not detected in cardiomyocytes (13-16).

The hallmarks of PKC activation by GPCRs and phorbol esters involve translocation to particulate/membrane structures followed by down-regulation (proteolysis) of the enzyme. Preliminary studies indicated that rPMT (400 ng/ml) does not detectably alter the partitioning of PKC isoforms between soluble and particulate fractions at early time points (30 min-2 hr, data not shown). PKC isoform abundance also was preserved at these early time points. However, given the very protracted kinetics for PLC activation by rPMT (which are quite distinct from the rapid kinetics for PLC activation by agonist-occupied GPCRs), subsequent studies examined the partitioning and abundance of PKC isoforms in cardiomyocyte cultures subjected to stimulation with rPMT for 24 hr.

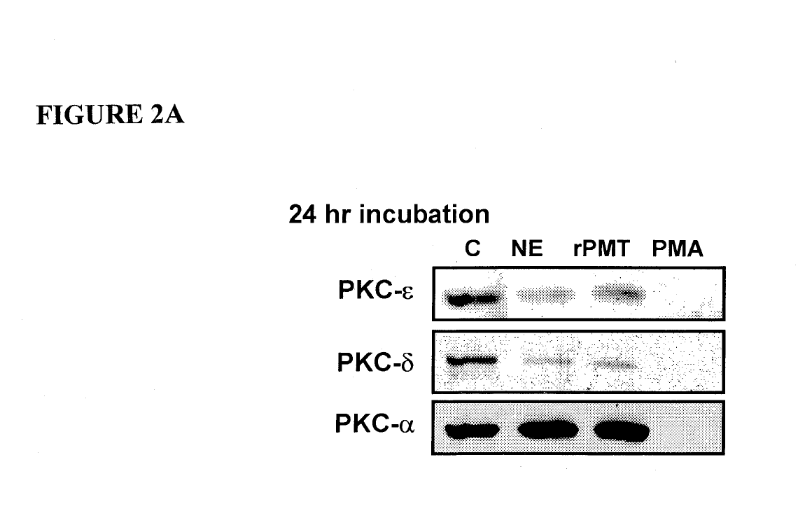

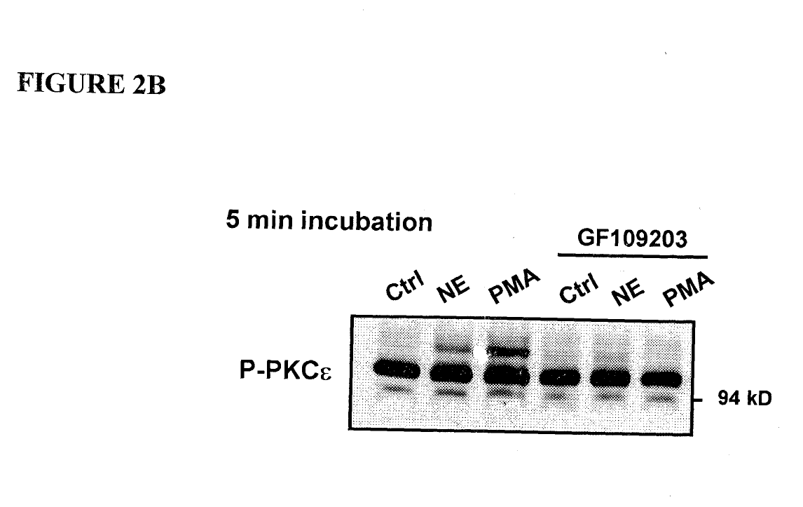

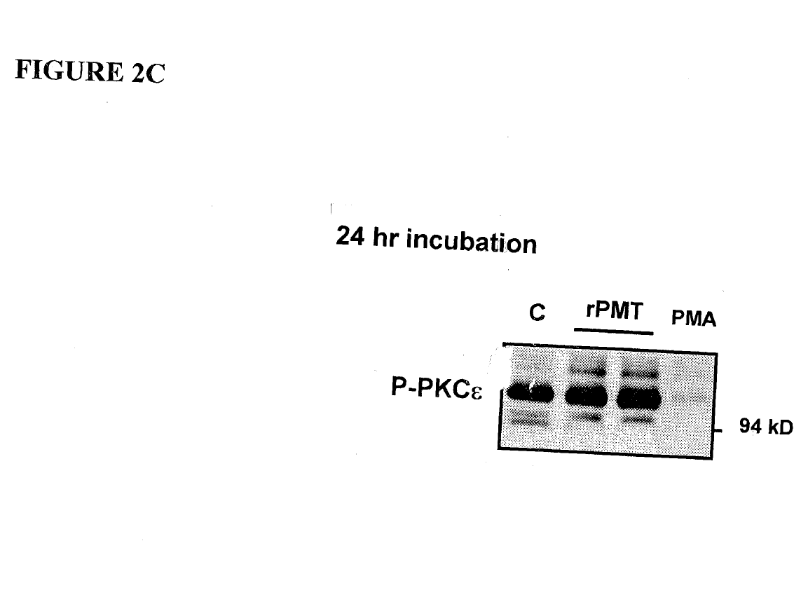

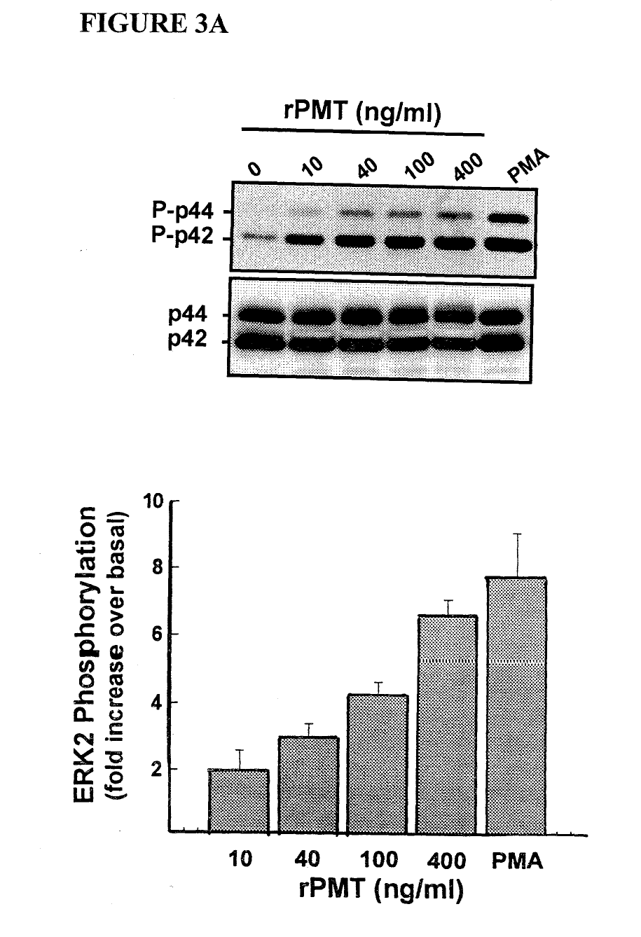

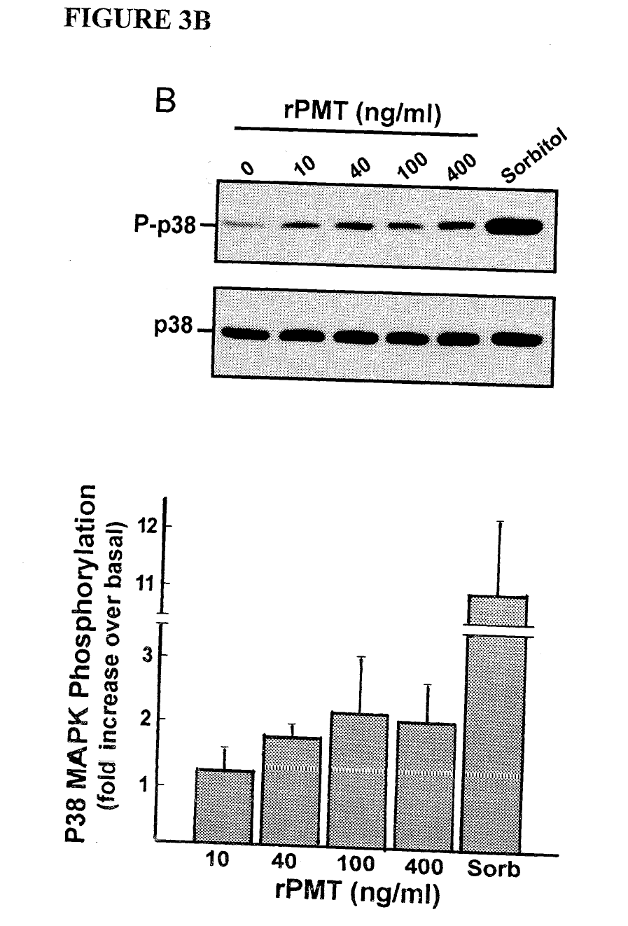

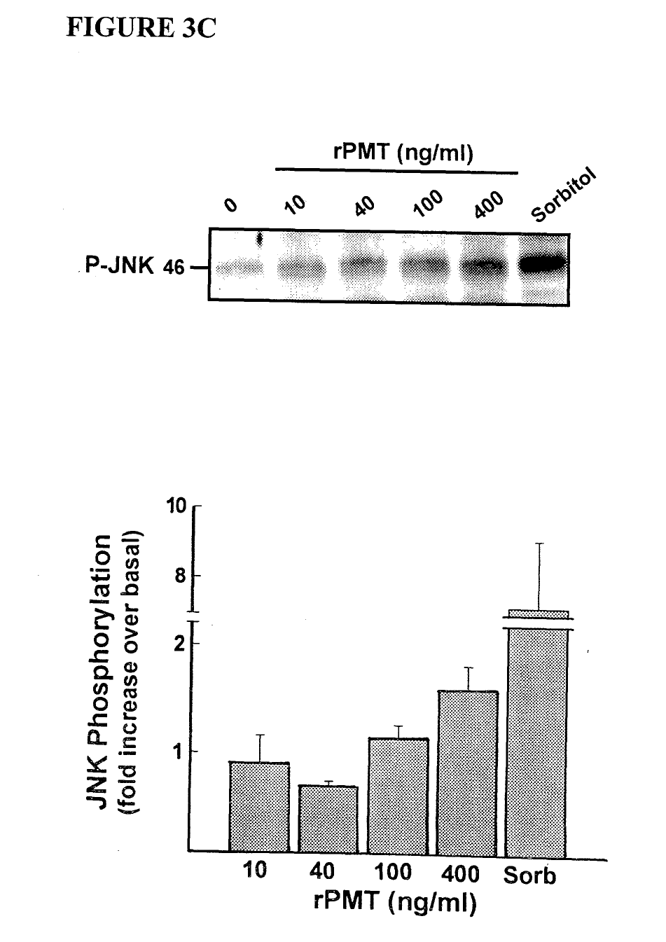

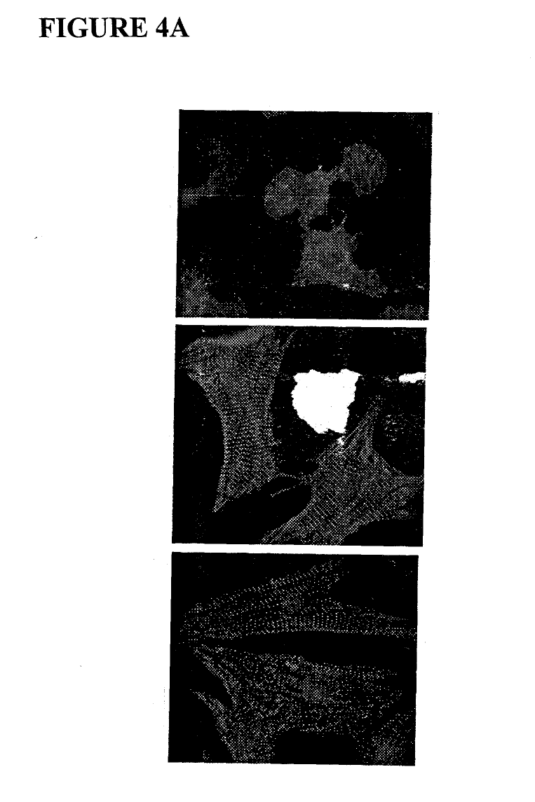

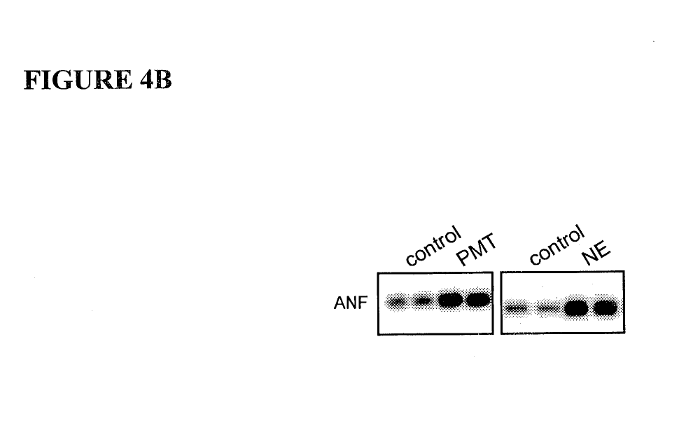

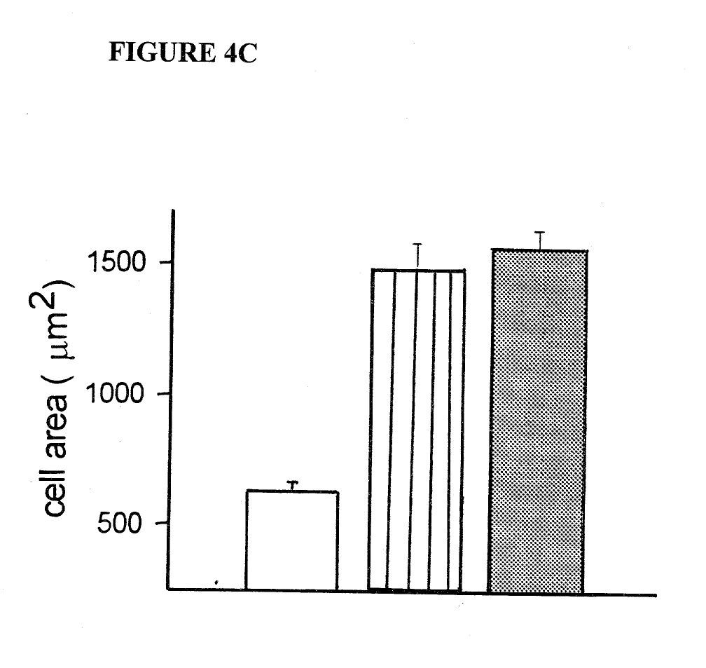

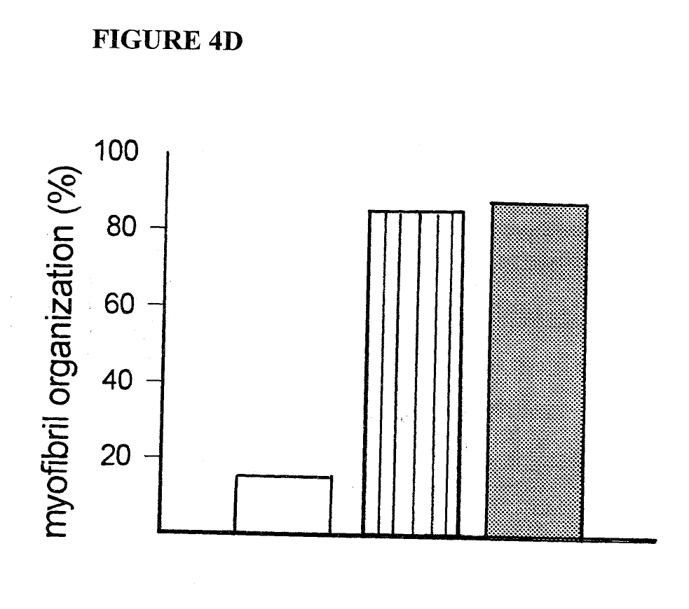

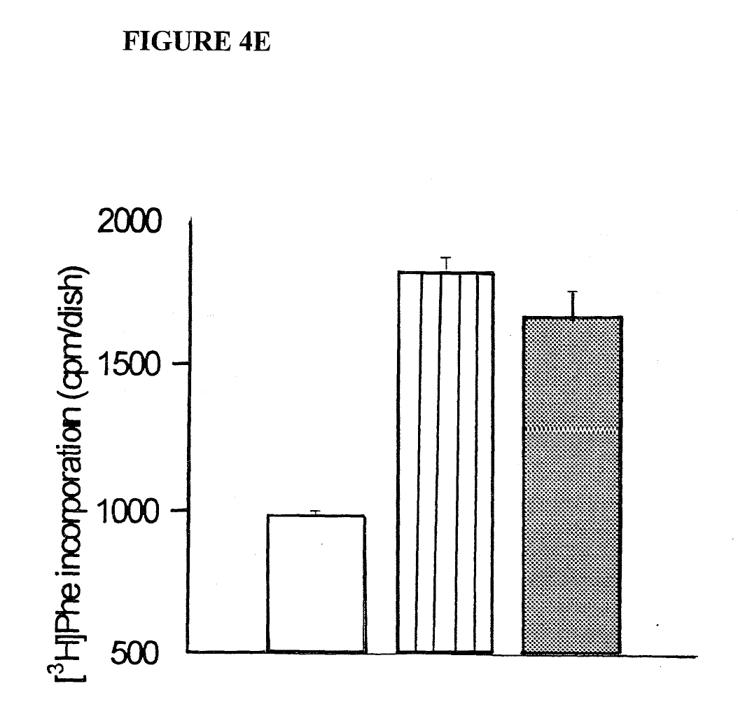

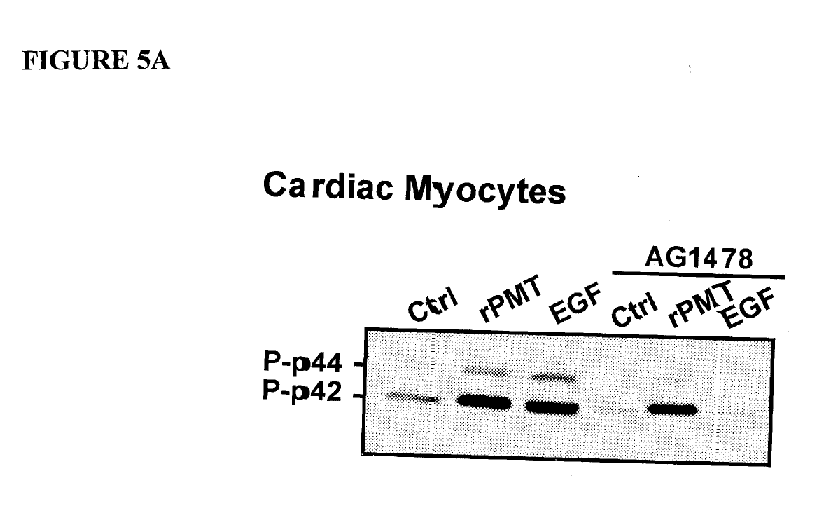

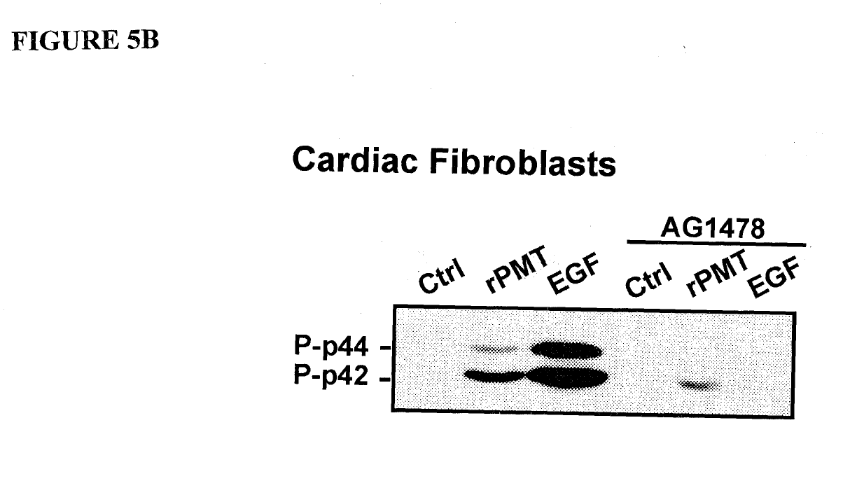

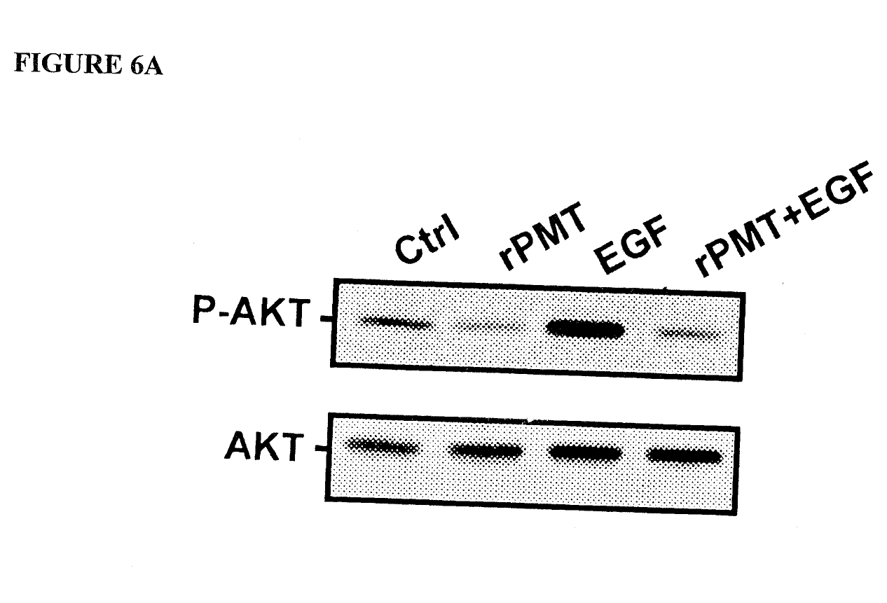

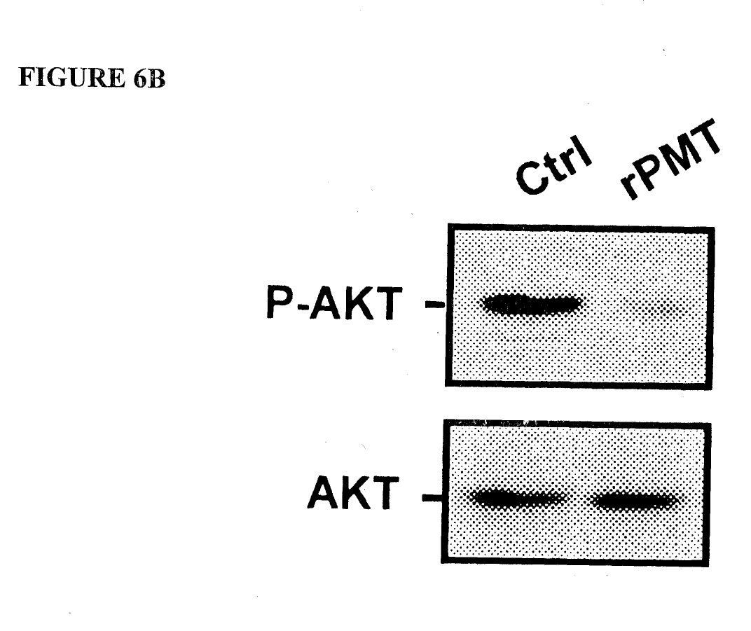

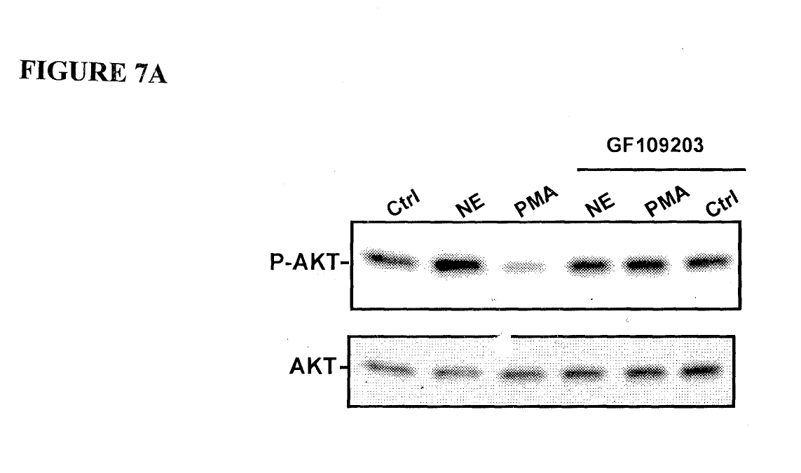

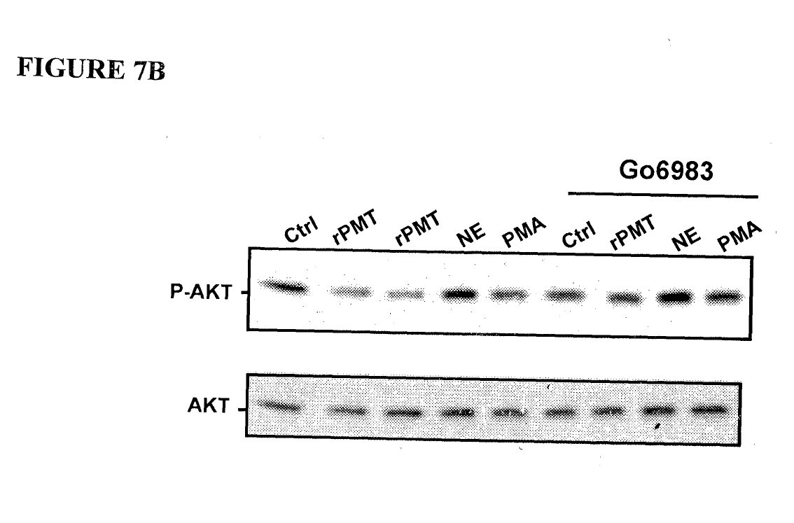

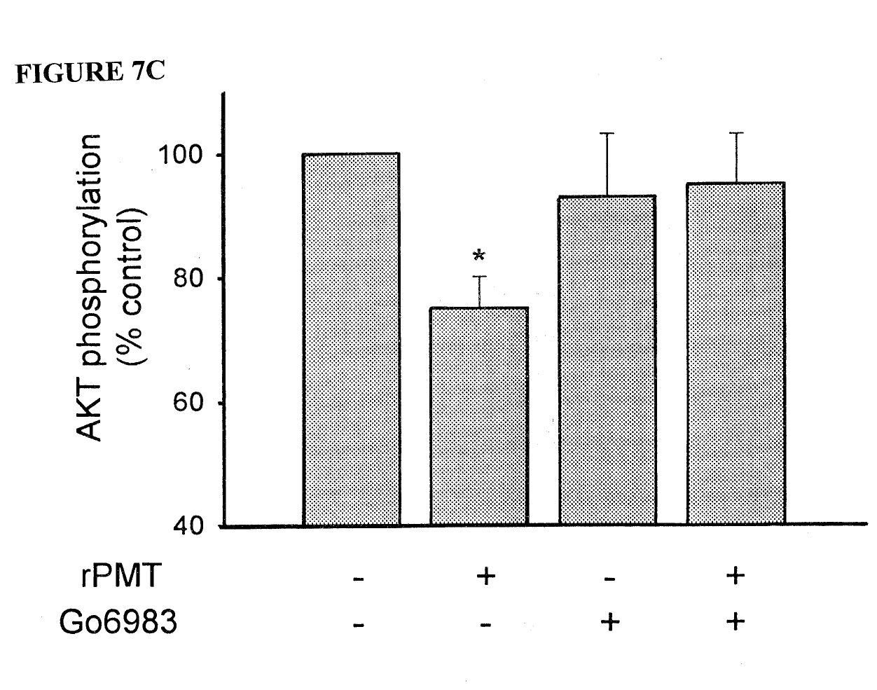

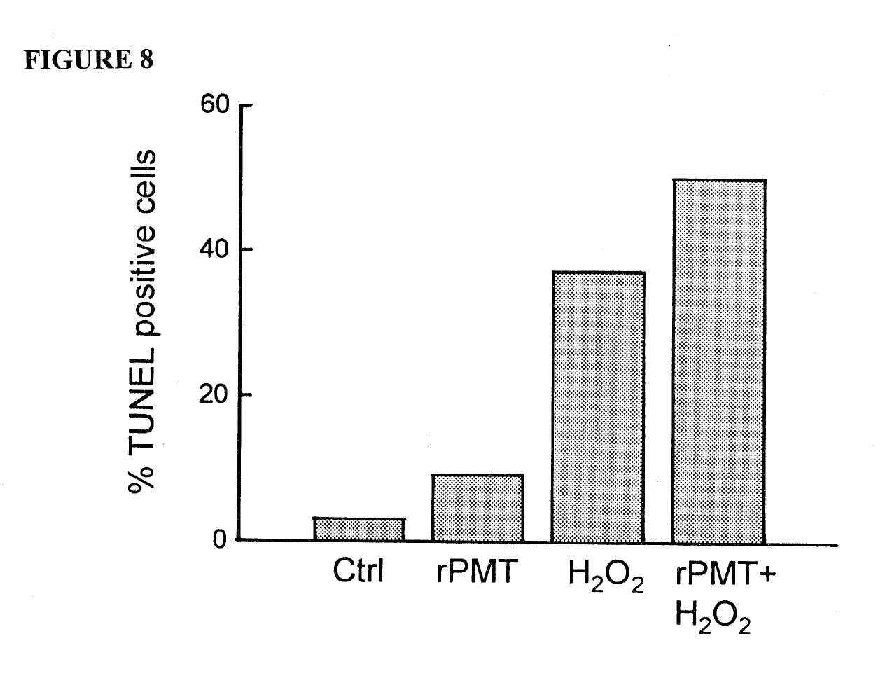

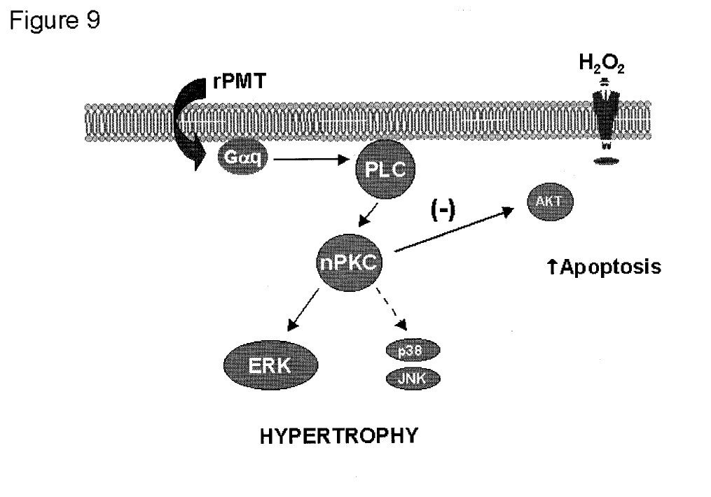

Here, persistent PKC activation is predicted to be detected as reduced amounts of enzyme that disproportionately associates with the particulate fraction, as detected in preparations from cardiomyocyte cultures exposed to the α1-adrenergic receptor agonist norepinephrine for 24 hr or in ventricles from mice that overexpress Gαq proteins (6, 13). Indeed, FIG. 2A shows that rPMT mimics the effect of norepinephrine to significantly reduce the abundance of PKCδ and PKCε in the soluble fraction. The abundance of PKC-δ and PKC-ε in the particulate fraction remains relatively preserved (data not shown). Separate experiments established that the rPMT-dependent decrease in PKCδ and PKC-ε abundance in the soluble fraction is not associated with the appearance of lower molecular weight degradation products. The concentration-response relationship for rPMT stimulation of PLC and activation of PKC coincide. Of note, persistent Gαcq stimulation, with rPMT or NE, targets to the activation of only the nPKC isoforms, PKCδ and PKCε. The abundance and subcellular distribution of PKCα and PKCλ is not altered by rPMT (or NE; FIG. 2A and data not shown).

Western blot analysis with an anti-phospho-PKC antibody was used as an independent measure of PKC activation. This antibody does not discriminate between PKC isoforms and would be predicted to detect bands with distinct mobilities for PKCα/PKCδ (82- and 78-kDa) and PKC-ε (96 kD). However, only a higher molecular mass species with a mobility that corresponds to PKC-ε was detected, including in cells acutely stimulated with PMA, where PKCα and PKCδ activation is readily detected by conventional methods (FIGS. 2B, 2C).

The failure to detect phosphorylated forms of PKCα and PKCδ could be due to differences in the relative expression levels of individual PKC isoforms (one to another) in cardiomyocytes, differences in the hybridization efficiency of this antiserum for individual PKC isoforms, or differences in the extent to which phosphorylation is involved in the activation process for individual PKC isoforms in cardiomyocytes. Nevertheless, PKC-ε phosphorylation/activation can be monitored with this antiserum. FIG. 2B shows that PKCε is detected as a single band in quiescent cardiomyocytes. PKC-ε is detected as this major immunoreactive band as well as a slower mobility, more-highly phosphorylated species in cells exposed to NE (10 μM for 5 min) or PMA (100 nM for 5 min). The appearance of the more highly phosphorylated species of PKC-ε in response to NE or PMA reflects PKC activation. The appearance of this band is prevented by the PKC inhibitor GF109203 (FIG. 2B, right). Separate experiments established that both the major more rapidly migrating band in quiescent cultures as well as the activation-dependent slower migrating (more phosphorylated) band are phosphorylated protein. Phospho-PKC immunoreactivity is completely stripped by acid phosphatase treatment (data not shown). FIG. 2C also shows that immunoreactivity is drastically reduced during pharmacologic down-regulation with PMA for 24 hrs, where there is proteolytic degradation of PKCε protein.

These results validate the use of the phospho-PKC antibody as a method to monitor PKC-ε activation. Hence, the effect of rPMT was examined. FIG. 2C shows that PKC-ε phosphorylation is enhanced in cardiomyocytes treated with rPMT.

Collectively, these studies support the conclusions that chronic signaling through Gαq leads to persistent activation of PKC, with selectivity for novel PKC isoforms.

The MAPK cascades that lie downstream in Gαq-dependent pathways in cardiomyocytes were next studied. FIG. 3 shows that rPMT induces dose-dependent increases in signaling through the ERK1/2, p38-MAPK, and JNK cascades, as detected by an increase in the phosphorylation of the terminal kinase of each pathway. For each, activation was observed with slow kinetics (detectable at 1 hr and increased progressively for 24 hr). Immunoblot analyses with antisera that recognize total (phosphorylated and non-phosphorylated) ERK1/2, p38-MAPK, and JNK enzymes show that rPMT does not significantly alter the expression of these proteins (FIG. 3A and B and data not shown). This indicates that the protracted kinetics for MAPK cascade activation by rPMT is not due to changes in terminal kinase protein expression. ERK1/2 activation by rPMT is quite robust. The magnitude of the response is comparable to acute stimulation with PMA. In contrast, JNK and p38-MAPK activation by rPMT is more modest. Responses are minor relative to the strong activation of JNK and p38-MAPK induced by sorbitol. rPMT displays potent growth-stimulatory properties in cells with proliferative potential and activates a spectrum of signaling molecules that individually have been implicated in the hypertrophic growth program. Therefore, the next studies examined the cellular actions of rPMT in cardiomyocytes.

FIG. 4 shows that rPMT induces all of the hallmarks of cardiomyocyte hypertrophy. rPMT mimics the effect of NE to promote myofibrillar organization, induce marked cellular enlargement, enhance [³H]phenylalanine incorporation, and induce ANF mRNA expression.

B. ERK Activation by rPMT does not Involve the EGF Receptor in Cardiomyocytes

There is extensive heterogeneity in the mechanisms for ERK cascade activation by GPCRs. Depending upon the cell type and particular GPCR, the ERK cascade can be activated by a Ras-independent pathway that involves PKC isoforms or a Ras-dependent pathway that is activated by receptor or non-receptor tyrosine kinases (EGF receptor family members, FAK, or the FAK-related kinase Pyk2). Seo et al. recently reported that rPMT stimulates the ERK cascade via a Ras-dependent (PKC-independent) pathway that involves EGF receptor transactivation in HEK293 cells (9). To determine whether this pathway mediates rPMT actions in cardiomyocytes, the sensitivity of rPMT-stimulated ERK activation to the EGF receptor-specific tyrphostin AG1478 was examined. FIG. 5A shows that AG1478 effectively blocks EGF receptor-mediated ERK phosphorylation, but induces only a very minor reduction in ERK phosphorylation by rPMT. As a control, similar studies were performed in cardiac fibroblasts. Here, rPMT-mediated activation of ERK displays a significant requirement for EGF receptor activity. AG1478 has a marked effect to prevent ERK activation by both EGF and rPMT (FIG. 5B).

These results indicate that Gαq stimulation leads to EGF receptor transactivation as a scaffold to assemble other proteins and phosphorylate ERK in cardiac fibroblasts, but the EGF receptor plays little role in Gαq signaling to ERK in cardiomyocytes. Consistent with this formulation, rPMT-dependent activation of ERK was markedly inhibited by the PKC inhibitor GF109203.

Inhibition of PKC also markedly attenuated rPMT-dependent stimulation of [³H]phenylalanine incorporation into proteins and completely abrogated the rPMT-dependent increase in total protein content. rPMT-dependent induction of these components of the hypertrophic phenotype was not inhibited by AG1478. These results identify a requirement for PKC isoforms in the rPMT signaling pathway.

C. rPMT Activation of PKC Leads to Negative Regulation of AKT; rPMT Enhances Susceptibility to H₂O₂-induced Apoptosis

Recent studies identify phosphatidylinositol (PI) 3-kinase-dependent activation of AKT as an important survival signal in cardiomyocytes (17). While most studies focus on mechanisms for AKT activation by receptor tyrosine kinases, AKT activation by GPCRs also has been reported [including in cardiomyocytes] (2, 18). The role of G protein α subunits in this process is disputed. Murga et al. identify Gαi-, Gαq-, and βγ dimer-dependent pathways for AKT activation (19). In contrast, studies by Bommakanti et al. implicate βγ dimers in AKT activation, but fail to identify AKT stimulation by either Gαi or Gαq. To the contrary, these investigators identify an effect of Gαq subunits to inhibit AKT (20). Given recent controversy pertaining to the mode of AKT modulation by Gαq, and the evidence that Gαq activation leads to enhanced apoptosis of cardiomyocytes, the studies next compared AKT protein expression and activation (detected as increased regulatory phosphorylation on Ser-473 in the C-terminal hydrophobic motif) under control conditions following stimulation with rPMT.

FIG. 6 shows that exposure to rPMT for 24 hr leads to reduced basal AKT phosphorylation. This effect was even more pronounced at 48 hr. The effect of EGF to promote AKT activation is robust in control cultures. This response is blunted in rPMT-treated cultures. Reduced levels of phospho-AKT in rPMT-treated cultures do not result from a change in AKT protein expression. Total AKT immunoreactivity is similar in control and rPMT-treated cultures.

Since there is recent evidence in tumor cells that certain PKC isoforms interact with AKT in a functionally relevant manner, and rPMT induces prominent PKC activation, the next studies examined whether PKC reduces AKT phosphorylation and mediates the effect of rPMT to negatively regulate AKT. FIG. 7A shows that acute PKC activation with PMA (but not by NE) leads to reduce basal AKT phosphorylation. Inhibitory modulation of AKT phosphorylation by PMA is prevented by the PKC inhibitor GF109203. FIGS. 7B and C show that AKT phosphorylation is reduced (relative to control cultures) following treatment with rPMT or PMA for 24 hr. Go6983 abrogates the negative regulation of AKT by rPMT, implicating PKC in this pathway. Go6983 (rather than GF109203) was used in these experiments carried out for 24 hr since it completely inhibited PMA-dependent down-regulation of PKC isoforms, whereas GF109203 did not. This could be due to differences in drug stability or other factors. It represents a technical factor that precludes the use of GF109203 in experiments with prolonged time courses. These results indicate that a phorbol ester- and Go6983-sensitive PKC isoform negatively regulates AKT. PKCδ is the only Go6983-sensitive isoform activated by rPMT. However, its role to link Gαq activation by rPMT to inhibitory regulation of AKT could not be evaluated further pharmacologically, as cardiomyocytes do not tolerate prolonged (24 hr) incubations with the PKC-δ -specific inhibitor rottlerin.

Decreased signaling through the AKT pathway could render cardiomyocytes vulnerable to stresses that induce apoptosis. Indeed, FIG. 8 shows that rPMT induces a small increase in the number of TUNEL-positive cells in cardiomyocyte cultures grown in serum-free conditions for 48 hr. H₂0₂ induces apoptosis and this response is augmented in rPMT-treated cultures.

III. Discussion

Previous attempts to identify the signaling properties of Gαq in cardiomyocytes relied largely on the molecular strategies widely used to isolate and discern the functional roles of other signaling molecules. These studies provided unanticipated evidence that transient Gαq overexpression is sufficient to induce changes that continue to drive cardiomyocyte hypertrophy long after the initiating stimulus has disappeared (21). This suggests a very important biological role for Gαq proteins but emphasizing the difficulty in discriminating direct effects of a transgene from secondary compensatory mechanisms that either drive or are the consequence of the pathology in transgenic models.

Another unanticipated finding of these studies was that Gαq stimulation induces a continuum of responses, from compensated hypertrophy to decompensated heart failure, as the stimulus strength increases or is maintained over prolonged intervals. While this suggests that hypertrophy and apoptosis may be initiated by a common Gαq-activated biochemical signal, a molecular signal that fulfills this requirement was not identified. Using rPMT as a pharmacologic Gαq agonist to identify signals that emanate from endogenous cardiomyocyte Gα_{q} proteins, studies reported herein indicate that nPKC isoforms represent such a mechanism.

Specifically, the major findings of this study are that Gαq stimulation by rPMT couples to the activation of PLC and nPKC isoforms, which lie upstream in the pathway(s) for activation of MAPK cascades (ERK, with lesser activation of JNK and p38-MAPK) and negative regulation of AKT (FIG. 9). rPMT is a potent in vitro stimulus for cardiomyocyte hypertrophy as a result of signaling through the nPKC/MAPK pathways. However, nPKC activation also represses the AKT pathway and prevents its recruitment by ligands that activate receptor tyrosine kinases, rendering the cardiomyocyte highly susceptible to stresses that induce apoptosis. This identifies a pivotal role for nPKC isoforms upstream in pathways that both promote hypertrophy and enhance the vulnerability of cardiomyocytes to inducers of apoptosis. According to this model, endogenous Gαq protein activation leads to cardiomyocyte hypertrophy, but even with intense stimulation, this does not lead to apoptosis under physiological conditions. However, cells that cannot recruit the AKT pathway poorly tolerate intervening insults that characterize disease progression.

In the context of the experimental models, this provides an appealing explaining for previous observations that transgenic overexpression of Gαq even at modest levels leads to a lethal dilated cardiomyopathy during the stress of pregnancy, parturition, or aortic banding (7). Similarly, this provides a novel mechanism to explain the progression from hypertrophy to cardiac decompensation that typifies clinical heart failure in humans.

Gq/11-coupled receptors employ divergent pathways to initiate signaling via the ERK cascade. This heterogeneity is dictated both by the identity of the GPCR as well as differences in the signaling machinery endogenous to various cell types. Seo et al. previously reported that rPMT stimulation of ERK requires transactivation of the EGF receptor in cultured HEK293 cells and studies herein detect a similar pathway for rPMT activation of ERK in cardiac fibroblasts. While “cross-talk” between Gq and EGF receptors is prominent in many of the experimental models typically used to define signal transduction mechanisms, the major pathway for rPMT activation of ERK in cardiomyocytes can be attributed to PKC. Since cardiomyocytes co-express multiple molecular forms of PKC, the identity of the isoform(s) in this pathway was examined. Using a reduced total level of immunoreactivity that preferentially localizes to the particulate fraction as a criterion for chronic PKC isoform activation, these studies identify nPKC isoforms PKCδ and PKCε (but not PKCα or PKCλ) as the isoforms activated during persistent Gαq activation (by rPMT or agonist-activated α1-receptors). These results are consistent with findings reported in mice that overexpress Gαq (6) and place nPKC isoforms in the pathway for rPMT activation of the ERK cascade. Since heart failure syndromes are characterized by persistent elevations of circulating catecholamines (and sustained increases in DAG), these chronic changes in nPKC localization and abundance (which impact on ERK and AKT and, to a lesser extent JNK and p38-MAPK signaling) are predicted to be particularly relevant to the pathophysiology of heart failure.

There is recent evidence that rPMT can act independently of the PLC/PKC pathway to activate ERK and other MAPK cascades in embryonic fibroblasts that lack Gα_{q} subunits (12). However, this does not appear to detract from the usefulness of rPMT as a reagent to define the signaling properties and cellular actions of endogenous Gαq subunits, as well as nPKC isoforms, in cardiomyocytes.

General mechanisms for growth factor-dependent activation of the PI-3K/AKT pathway, and their contribution to cell survival, proliferation and differentiation, have been the focus of considerable recent research interest. While growth responses reflect the integrated and dynamic balance between stimulatory and inhibitory pathways, mechanisms that curtail AKT activation are less well understood, and there is only very limited information on AKT regulation by PKC isoforms.

Studies reported herein identify an effect of PMA and rPMT to negatively regulate AKT phosphorylation in cardiomyocytes, with inhibitory regulation most readily attributable to a nPKC isoform(s). Results that place PKC in a pathway that negatively impacts on cardiomyocyte survival were not anticipated.

PKC is a well-recognized mediator of ischemic preconditioning and is generally viewed as cardioprotective. However, results reported herein suggest that PKC isoforms fulfill a range of cellular actions with very distinct biological consequences. PKC is highly protective when acutely activated in the course of ischemia/reperfusion injury. However, chronic PKC activation by catecholamines may repress survival pathways and be deleterious to the natural history of heart failure. These disparate effects of PKC may result from the actions of a single, overlapping, or distinct PKC isoforms.

There is recent evidence that inhibitory modulation of AKT by PKC, which limits growth factor signaling, is a general phenomenon. There is recent evidence that PKCζ (but not PKCα or PKCδ) co-immunoprecipitates with AKT and attenuates the phosphorylation of AKT and its downstream target p70^{s6k} in CHO and breast cancer cells (22, 23). Other studies identify an effect of PMA to attenuate IGF-1-dependent activation of AKT in PC12 cells. Studies with pharmacologic inhibitors identify PKCδ as the isoform that negatively regulates AKT in PC12 cells. The mechanism for negative regulation is presumed to be related to the physical association that can be demonstrated between PKCδ and the PH-domain of AKT (24, 25). However, other mechanisms involving PKC-dependent changes in the phosphorylation of other components of the PI-3K signaling cascade and the action of phosphatases have been proposed but are not fully understood (26).

Finally, the implications of the negative regulation of AKT by PKC isoforms in the context of cardiomyocyte activation by Gq-coupled receptors deserve comment.

GPCRs activate an array of signaling mechanisms that influence the growth, survival, and biological properties of cardiomyocytes. Receptor tyrosine kinases (such as the epithelial growth factor [EGF] receptor) activate the PI-3K/AKT pathway as a mechanism to impact favorably on cell survival and critically influence various metabolic responses.

Recent studies in model systems identify a mechanism whereby GPCRs transactivate the EGF receptor. Recent studies in cardiomyocytes identify a rather modest level of AKT activation (relative to IGF-1) by PAR-1, which couples to Gq and other G proteins (2). This mechanism has never been identified in the heart.

The studies herein (data not shown) provide novel evidence that certain GPCRs (in particular, protease-activated receptor-1 [PAR-1]) transactivate the EGF receptor in cardiomyocytes. Of note, transactivation of the EGF receptor by PAR-1 is repressed by PKC, and is revealed by treatment of cells with PKC inhibitors. Insofar as EGF receptor transactivation provides a strong stimulus for AKT activation, this identifies a novel target for therapeutic intervention in heart failure. According to this formulation, the effects of PKC inhibitors to augment PAR-1 transactivation of the EGF receptor (and hence activation of AKT) identifies an additional therapeutic role for PKC inhibitors in the context of regimens to delay the progression of pathological changes in heart failure.

References

1. Hilal-Dandan, R., Ramirez, M. T., Villegas, S., Gonzalez, A., Endo-Mochizuki, Y., Brown, J. H., and Brunton, L. L. 1997. Endothelin ETA receptor regulates signaling and ANF gene expression via multiple G protein-linked pathways. Am. J. Physiol. 272: H130-H137.

2. Sabri, A., Muske, G., Zhang, H., Pak, E., Darrow, A., Andrade-Gordon, P., and Steinberg, S. F. 2000. Signaling properties and functions of two distinct cardiomyocyte protease-activated receptors. Circ. Res. 86: 1054-1061.

3. Dorn, G. W. and Brown, J. H. 1999. Gq signaling in cardiac adaptation and maladaptation. Trends Cardiovasc. Med. 9: 26-34.

4. LaMorte, V. J., Thorburn, J., Absher, D., Spiegel, A. M., Brown, J. H., Chien, K. R., Feramisco, J. R., and Knowlton, K. U. 1994. G_{q}- and ras-dependent pathways mediate hypertrophy of neonatal rat ventricular myocytes following αₗ-adrenergic stimulation. J. Biol. Chem. 269: 13490-13496.

5. Akhter, S. A., Luttrell, L. M., Rockman, H. A., Iaccarino, G., Lefkowitz, R. J., and Koch, W. J. 1998. Targeting the receptor-G_{q} interface to inhibit in vivo pressure overload myocardial hypertrophy. Science 280: 574-577.

6. D'Angelo, D. D., Sakata, Y., Lorenz, J. N., Boivin, G. P., Walsh, R. A., Liggett, S. B., and Dorn, G. W. 1997. Transgenic Gαq overexpression induces cardiac contractile failure in mice. Proc. Natl. Acad. Sci. USA 94: 8121-8126.

7. Adams, J. W., Sakata, Y., Davis, M. G., Sah, V. P., Wang, Y., Liggett, S. B., Chien, K. R., Brown, J. H., and Dorn, G. W. 1998. Enhanced Gαq signaling: A common pathway mediates cardiac hypertrophy and apoptotic heart failure. Proc. Natl. Acad. Sci. USA 95: 10140-10145.

8. Mende, U., Kagen, A., Meister, M., and Neer, E. J. 1999. Signal transduction in atria and ventricles of mice with transient cardiac expression of activated G protein αq . Circ. Res. 85: 1085-1091.

9. Seo, B., Choy, E. W., Maudsley, S., Miller, W. E., Wilson, B. A., and Luttrell, L. M. 2000. Pasteurella multocida toxin stimulates mitogen-activated protein kinase via G_{q/11}-dependent transactivation of the epidermal growth factor receptor. J. Biol. Chem. 275: 2239-2245.

10. Lacerda, H. M., Lax, A. J., and Rozengurt, E. 1996. Pasteurella multocida toxin, a potent intracellularly acting mitogen, induces p125^{FAK} and paxillin tyrosine phosphorylation, actin stress fiber formation, and focal contact assembly in Swiss 3T3 cells. J. Biol. Chem. 271:4 39-445.

11. Wilson, B. A., Zhu, X., Ho, M., and Lu, L. 1997. Pasteurella multocida toxin activates the inositol triphosphate signaling pathway in Xenopus oocytes via G_{q}α-coupled phospholipase C-β1. J. Biol. Chem. 272: 1268-1275.

12. Zywietz, A., Gohla, A., Schmelz, M., Schultz, G., and Offermanns, S. 2000. Pleiotropic effects of Pasteurella multocida toxin are mediated by Gq-dependent and -independent mechanisms. Involvement of G_{q} but not G₁₁. J. Biol. Chem. 276(6): 3840-3845.

13. Rohde, S., Sabri, A., Kamasamudran. R., and Steinberg, S. F. 2000. The α₁-adrenergic receptor subtype- and protein kinase C isoform-dependence of norepinephrine's actions in cardiomyocytes. J. Mol. Cell. Cardiol. 32: 1193-1209.

14. Rybin, V. O. and Steinberg, S. F. 1994. Protein kinase C isoform expression and regulation in the developing rat heart. Circ. Res. 74: 299-309.

15. Disatnik, M. H., Buraggi, G., and Mochly-Rosen, D. 1994. Localization of protein kinase C isozymes in cardiac myocytes. Experimental Cell Research 210: 287-297.

16. Puceat, M., Hilal-Dandan, R., Strulovici, B., Brunton, L. L., and Brown, J. H. 1994. Differential regulation of protein kinase C isoforms in isolated neonatal and adult cardiomyocytes. J. Biol. Chem. 269: 16938-16944.

17. Fujio, Y., Nguyen, T., Wencker, D., Kitsis, R. N., and Walsh, K. 2000. Akt promotes survival of cardiomyocytes in vitro and protects against ischemia-reperfusion injury in mouse heart. Circulation 101: 660-667.

18. Chesley, A., Lundberg, M. S., Asai, T., Xiao, R. P., Ohtani, S., Lakatta, E. G., and Crow, M. T. 2000. The β-adrenergic receptor delivers an anti-apoptotic signal to cardiac myocyte through G₁-dependent coupling to phosphatidylinositol 3′-kinase. Circ. Res. 87: in press.

19. Murga, C., Laguinge, L., Wetzker, R., Cuadrado, A., and Gutkind, J. S. 1998. Activation of Akt/protein kinase B by G protein-coupled receptors. A role for α and βγ subunits of heterotrimeric G proteins acting through phosphatidylinositol-3-OH kinase γ. J. Biol. Chem. 273: 19080-19085.

20. Bommakanti, R. K., Vinayak, S., and Simonds, W. F. 2000. Dual regulation of Akt/Protein kinase B by heterotrimeric G protein subunits. J. Biol. Chem. 275: 38870-38876.

21. Mende, U., Kagen, A., Cohen, A., Aramburu, J., Schoen, F. J., and Neer, E. J. 1998. Transient cardiac expression of constitutively active Gαq leads to hypertrophy and dilated cardiomyopathy by calcineurin-dependent and independent pathways. Proc. Natl. Acad. Sci. USA 95: 13893-13898.

22. Doornbos, R. P., Theelen, M., van der Hoeven, P. C., van Blitterswijk, W. J., Verkleij, A. J., and van Bergen en Henegouwen, P. M. 1999. Protein kinase Cζ is a negative regulator of protein kinase B activity. J. Biol. Chem. 274: 8589-8596.

23. Mao, M., Fang, X., Lu, Y., Lapushin, R., Bast, R. C., and Mills, G. B. 2000. Inhibition of growth-factor-induced phosphorylation and activation of protein kinase B/Akt by atypical protein kinase C in breast cancer cells. Biochem. J. 352 Pt 2: 475-482.

24. Konishi, H., Matsuzaki, H., Tanaka, M., Ono, Y., Tokunaga, C., Kuroda, S., and Kikkawa, U. 1996. Activation of RAC-protein kinase by heat shock and hyperosmolarity stress through a pathway independent of phosphatidylinositol 3-kinase. Proc. Natl. Acad. Sci. USA 93:7639-7643.

25. Zheng, W. H., Kar, S., and Quirion, R. 2000. Stimulation of protein kinase C modulates insulin-like growth factor-1- induced AKT activation in PC12 cells. J. Biol. Chem. 275: 13377-13385.

26. Meier, R., Thelen, M., and Hemmings, B. A. 1998. Inactivation and dephosphorylation of protein kinase Bα (PKBα) promoted by hyperosmotic stress. EMBO J. 17: 7294-7303.

27. Busuttil, S. J., Morehouse, D. L., Youkey, J. R., Singer, H. A. 1996. Antisense suppression of protein kinase C-alpha and -delta in vascular smooth muscle. J. Surg. Res. 63(1): 137-142.

28. Molkentin, J., Dorn, II G. 2001. Cytoplasmic signaling pathways that regulate cardiac hypertrophy. Annu. Rev. Physiol. 63: 391-426.

29. Frey, N., McKinsey, T. A., Olson, E. N . 2000. Decoding calcium signals involved in cardiac growth and function. Nat. Med. 6(11): 1221-7.

30. Molkentin, J. D. 2000. Calcineurin and beyond: cardiac hypertrophic signaling. Circ. Res. 87(9): 731-8.

31. Bolli, R. 2000. The late phase of preconditioning. Circ. Res. 87(11): 972-83.

32. Simkhovich, B. Z., Pryklenk, K., Kloner, R. A. 1998. Role of protein kinase C as a cellular mediator of ischemic preconditioning a critical review. Cardiovasc. Res. 40(1): 9-22.

33. Chen, C., Mochly-Rosen, D. 2001. Opposing Effects of delta and xi PKC in Ethanol-induced Cardioprotection. J. Mol. Cell. Cardiol. 33(3): 581-5.

34. Chen, C. H., Gray, M. O., Mochly-Rosen, D. 1999. Cardioprotection from ischemia by a brief exposure to physiological levels of ethanol: role of epsilon protein kinase C. Proc. Natl. Acad. Sci. USA. 96(22): 12784-9.

35. Mochly-Rosen, D., Wu, G., Hahn, H., Oninska, H., Liron, T., Lorenz, J. N., Yanti, A., Robbins, 2nd. 2000. Cardiotrophic effects of protein kinase C epsilon: analysis by in vivo modulation of PK Cepsilon translocation. Cir. Res. 86(11): 1173-9.

36. Fleming, I., MacKenize, S. J., Vernon, R. G., Anderson, N. G., Houslay, M. D., Kilgour, E. 1998. Protein kinase C isoforms play differential roles in the regulation of adipocyte differentiation. Biochem. J. 333(Pt 3): 719-27.

37. MacKenzie, S., Fleming, I., Houslay, M. D., Anderson, N. G., Kilgour, E. 1997. Growth hormone and phorbol esters require specific protein kinase C is mitogen-activated protein kinases in 3T3-F442A cells. Biochem. J. 324(Pt 1): 159-65.

38. Sil, P., Kandaswamy, V., Sen, S. 1998. Increased protein kinase C activity in myotrophin-induced myocyte growth. Circ. Res. 82(11): 1173-88.

What is claimed is:

1. A method of inhibiting the onset of a cardiac disorder in a subject afflicted with cardiac hypertrophy, comprising administering to the subject a prophylactically effective amount of an agent that specifically reduces the activity of PKC-δ or PKC-ε present in the subject's cardiomyocytes.

2. The method of claim 1, wherein the cardiac disorder is selected from the group consisting of hypertensive heart disease, valvular heart disease, diabetic heart disease and ischemic heart disease.

3. A method of reducing the activity of PKC-δ or PKC-ε present in cardiomyocytes of a subject afflicted with cardiac hypertrophy, comprising administering to the subject an effective amount of an agent that specifically reduces the activity of PKC-δ or PKC-ε present in the subject's cardiomyocytes.

4. The method of claim 1 or 3, wherein the subject is selected from the group consisting of a mouse, a rat, a dog, a guinea pig, a ferret, a rabbit and a primate.

5. The method of claim 4, wherein the subject is a human.

6. The method of claim 1 or 3, wherein the agent specifically inhibits PKC-δ.

7. The method of claim 1 or 3, wherein the agent specifically inhibits PKC-ε.

8. The method of claim 1 or 3, wherein the agent is a nucleic acid or a polypeptide.

9. The method of claim 1 or 3, wherein the agent is rottlerin.

10. A method of reducing the activity of PKC-δ or PKC-ε in a hypertrophic cardiomyocyte, comprising contacting the cardiomyocyte under suitable conditions with an agent that specifically reduces the activity of PKC-δ or PKC-ε present therein.

11. The method of claim 10, wherein the agent specifically inhibits PKC-δ.

12. The method of claim 10, wherein the agent specifically inhibits PKC-ε.

13. The method of claim 10, wherein the agent is a nucleic acid or a polypeptide.

14. The method of claim 10, wherein the agent is rottlerin.

15. An article of manufacture comprising (a) an agent that specifically reduces the activity of PKC-δ or PKC-ε present in a cardiomyocyte, and (b) instructions for using the agent to inhibit the onset of a cardiac disorder in a subject afflicted with cardiac hypertrophy.

16. The article of manufacture of claim 15, further comprising a pharmaceutically acceptable carrier.
